(19) United States
(12) Patent Application Publication
(10) Pub. No.: US 2022/0409186 A1
(43) Pub. Date: Dec. 29, 2022

(54) SYSTEM AND METHOD FOR ESTIMATING A FERTILITY STATUS OF A WOMAN

(71) Applicant: AVA AG, Zürich (CH)

(72) Inventor: Mohaned SHILAIH, Zürich (CH)

(21) Appl. No.: 17/753,251

(22) PCT Filed: Aug. 28, 2020
(86) PCT No.: PCT/EP2020/074052

§ 371 Date: Feb. 24, 2022

(30) Foreign Application Priority Data
Aug. 29, 2019 (EP) 19194324.0
Feb. 17, 2020 (EP) 20157786.3

(51) Int. Cl. A61B 10/00; G16H 40/63; A61B 5/00; A61B 5/11; A61B 5/0205

(52) U.S. Cl. CPC A61B 10/0012; G16H 40/63; A61B 5/7264; A61B 5/1118; A61B 5/02055; A61B 2010/0019; A61B 2010/0016

(57) ABSTRACT

The invention relates to a system (4) for estimating a fertility status of a woman, particularly for determining a conception probability of a woman, the system (4) comprising:—A wearable device (2A, 2B), comprising at least one sensor (201, 203, 204) configured to record at least one physiological signal from a woman wearing the wearable device (2A, 2B) and to generate sensor data from the at least one physiological signal, wherein the wearable device (2A, 2B) is configured and arranged to provide the sensor data to—An evaluation system (1) configured and arranged to receive and process the sensor data from the wearable device (2A, 2B), wherein the evaluation system (1) is further configured and arranged to classify the sensor data into at least a first group and a second group, wherein the first group is associated to sensor data indicative of a woman having a high fertility status and wherein the second group is associated to sensor data indicative of a woman having a low fertility status.

The invention relates to a system and a computer-implemented method of estimating a fertility status of a woman with a wearable computerized device.

For conception and pregnancy, a complex interaction of hormones from the ovaries, pituitary gland and the hypothalamus that governs the female menstrual cycle is necessary. For successful pregnancy and conception, a vast number of hormones needs to be synchronized by the body of the woman in amount and time. For certain hormones involved in the menstrual cycle and conception, such as progesteron or oestrogen, it is known that if these hormones are not synchronized, conception and pregnancy is not possible.

Given the degree of complexity of positive and negative feedback loops between the hormones, it is not surprising that hormonal imbalances are often the underlying cause of female infertility or sub-fertility. Though, as long as the menstrual cycle is perceived as regular, hormonal imbalances will not be noticed by the woman nor by her health care practitioner. Only when the woman has tried to conceive for months or even years without success, a hormonal analysis will discover the imbalance.

It is therefore an unmet need to provide a system for an early estimation of a fertility status of a woman; the fertility status being indicative for a woman being fertile, sub-fertile or clinically infertile, particularly in the context of the above-mentioned hormonal imbalances.

The fertility status can for example be considered as a probability measure indicating whether the chances of a woman getting pregnant are low or high.

A system and a method providing such a fertility status estimation could help couples seek advice from a health care practitioner much earlier, thereby, shortening the time to pregnancy. In cases, when the woman does not conceive, despite her chances of getting pregnant being predicted as high, i.e. having a high fertility status, such a system might also help identify sub-fertility causes that are non-hormonal, such as e.g. blocked fallopian tubes, also known as tubal factor infertility. Male sub-fertility might be another reason, why a woman, whose fertility status is high, does not conceive.

It is an object of the present invention to provide a method and system for predicting whether the chances of a female to conceive are low or high, i.e. whether the fertility status is high or not.

According to the present invention, these objects are achieved through the features of the independent claims. In addition, further advantageous embodiments can be found in the dependent claims and the description.

The objective can be achieved by recording physiological signals during one or more menstrual cycles, processing the sensor data generated form the signal and analysing the sensor data accordingly. The object can particularly be achieved by performing a similarity analysis that compares the sensor data acquired from the woman to reference sensor data from a group of women having a high fertility status and/or a group of females having a low fertility status, i.e. being sub-fertile or infertile.

### Definitions

The term ‘operationally connected’ particularly denotes a computerized network connection or communication via a network, e.g. a cellular network, wireless network such as radio, Bluetooth or WiFi, a wired network such a Local Area Network (LAN) or Wide Area Network (WAN), as well as a connection via the internet.

The term “computerized device” or “computerized system” or a similar term denotes one or more devices comprising one or more processors operable or operating according to one or more computer programs.

The term “mobile device” and similar terms particularly relate to a small computer, particularly small enough to hold and operate in the hand and having an operating system capable of running mobile apps—computer programs designed to run on mobile devices.

A mobile device, such as a mobile phone, a smart phone, a smart watch, a portable music player or a tablet computer, particularly comprises at least one processor, the so-called CPU (central processing unit). Furthermore a mobile device particularly comprises means for cellular network connectivity for connecting to a mobile network, such as for example GSM (Global System for Mobile Communications), 3G, 4G or 5G, CDMA (Code Division Multiple Access), CDMA2000, UTMS (Universal Mobile Telecommunications System), or LTE (Long Term Evolution).

The mobile device might comprise a display screen with a small numeric or alphanumeric keyboard or a touchscreen configured as an interface. The mobile device is particularly configured to connect to the Internet and interconnect with other computerized devices via Wi-Fi, Bluetooth or near field communication (NFC). Integrated cameras, digital media players, mobile phone and GPS capabilities are common.

The mobile device is particularly configured to provide a time reference for apps and sensor data, wherein said time reference is provided by a time reference system such as an internal clock or timer of the mobile device or by external signals that are for example received by the internet, the mobile network, or GPS. The time reference can be adjusted for different time zones.

The terms ‘processor’ or ‘computer’, or system thereof, are used herein as ordinary context of the art, such as a general purpose processor or a micro-processor, RISC processor, or DSP, possibly comprising additional elements such as memory or communication ports. Optionally or additionally, the terms ‘processor’ or ‘computer’ or derivatives thereof denote an apparatus that is capable of carrying out a provided or an incorporated computer program and/or is capable of controlling and/or accessing data storage apparatus and/or other apparatus such as input and output ports. The terms ‘processor’ or ‘computer’ denote also a plurality of processors or computers connected, and/or linked and/or otherwise communicating, possibly sharing one or more other resources such as a memory.

As used herein, the terms ‘server’ denotes a computerized device providing data and/or operational service or services to one or more other computerized devices or computers.

The terms ‘computer program’, ‘application’ or ‘app’ may be used interchangeably according to the context thereof, and denote one or more instructions or directives or circuitry for performing a sequence of operations that generally represent an algorithm and/or other process or method. The computer program is stored in or on a medium such as RAM, ROM, or disk, or embedded in a circuitry accessible and executable by an apparatus such as a processor or other circuitry.

The processor and computer program may constitute the same apparatus, at least partially, such as an array of electronic gates, such as FPGA or ASIC, designed to perform a programmed sequence of operations, optionally comprising or linked with a processor or other circuitry.

A device for storing and/or comprising a program and/or a data storage particularly constitutes an article of manufacture. The computer program and/or the data can be stored in or on a non-transitory medium.

According to claim 1 a system, particularly a computerized system for estimating or determining a fertility status of a woman, particularly for determining a conception probability of a woman, comprises at least the following components:
A wearable, particularly computerized device, comprising at least one sensor configured to record at least one physiological signal from a woman wearing the wearable device and to generate sensor data from the at least one physiological signal, wherein the wearable device is configured and arranged to provide the sensor data to
An evaluation system configured and arranged to receive and process the sensor data from the wearable device, wherein the evaluation system is further configured and arranged to classify the sensor data into at least a first group and a second group, wherein the first group is associated to sensor data indicative of a woman having a high fertility status and wherein the second group is associated to sensor data indicative of a woman having a low fertility status.

The term “computerized” particularly refers to a system or a device comprising one or more processors operable or operating according to one or more programs.

The term “wearable device” refers to a device that has a weight and dimension that allow a person to carry the device for essentially any time interval. The wearable device can have similar properties to a mobile device and particularly can be worn while not being held in the hand.

The wearable device is particularly a body-wearable device having adjustment means to fix the device to a body part of the person.

The wearable device can comprise or consist of two or more physically separated devices; one or more wearable sensor devices and a portable electronic device.

The wearable device is or comprises particularly a wrist-wearable device having adjustment means, such as an adjustable wrist band, e.g. a bracelet or a cuff, to fix the device to a wrist or a joint of the person.

The wearable device can be or comprise any item that has contact to the skin, such as but not limited to, a watch-like device worn on the wrist, a bracelet or a cuff worn on the body, a ring or a device or clamp worn on the fingertip, or an in-ear device. A wearable device can also relate to components being integrated in a shirt or other garment.

The wearable device is particularly configured to be worn such that skin contact of the wearable device is facilitated, when the device is worn by a woman, such that physiological signals can be recorded.

The wearable device might comprise a computer. The wearable device can be a smart watch.

A physiological signal is essentially any signal that has its origin by the woman's physiology and that varies in a correlated fashion with the menstrual cycle of the woman.

The term “sensor data” particularly refers to digital data that are generated from the physiological signal. As such the senor data can be digitally sampled data directly representing a temporal evolution of the physiological signal. Alternatively or additionally, the sensor data can be processed with additional filters or filter functions and still be referred to as sensor data in the context of the current specification.

The wearable device generating the sensor data is further configured to provide the sensor data to an evaluation system.

The evaluation system can be fully comprised, particularly physically integrated in the wearable device, partially comprised by the wearable device or arranged completely externally of the wearable device and physically, particularly mechanically disconnect.

Thus, particularly in case the evaluation system is mechanically disconnect to the wearable device, the wearable device as well as the evaluation system can comprise a communication system for transmitting the sensor data to the evaluation system, i.e. the provision of sensor data can be facilitated by means of the communication systems.

The evaluation system is configured and arranged to receive and process the sensor data, i.e. the evaluation system can comprise a computer program being executable on one or more processors of the evaluation system.

The computer program can be stored on an external server or stored on a memory device of the evaluation system.

The computer program comprises computer program code configured to cause the evaluation system to execute the necessary steps to perform the processing particularly the classification of the sensor data.

The classification of the sensor data particularly comprises assigning the sensor data to at least the first or to the second group.

Beside the first and the second group there might be one or more other groups into which the evaluation system can classify the sensor data.

For example, a third group into which classification can take place is indicative of sensor data of a woman having a medium fertility status indicating a conception probability between the high and the low fertility status.

Alternatively or in addition, a third respective fourth group into which classification can take place might be indicative for a non-estimated or not yet estimated fertility status or an unknown or unclear fertility status.

The first group might be associated to sensor data that are not classified into the second group, i.e. classification into the first group takes place as a consequence of not being classified into the second group rather than the classification to the first group takes due to a positive verifying. Similarly, the second group might be associated to sensor data that are not classified into the first group.

The first and the second group and particularly additional groups each are indicative to a distinct estimated fertility status.

The fertility status particularly refers to a probability measure or a probability indicating whether the chances of a woman getting pregnant are low or high.

The fertility status is particularly not a status that is indicative of a period during the menstrual cycle, when conception is most likely, i.e. some 5 days before until one or two days after ovulation. Thus, the fertility status is particularly not suited for fertility awareness.

For this reason, the fertility status comprises information about the general capability of conceiving during the next month.

The fertility status does particularly not vary significantly, i.e. to a degree that fertile days during the menstrual cycle can be identified during a menstrual cycle.

The fertility status can indicate that a woman is clinically infertile. Such a fertility status would at least correspond to a low fertility status.

The term “clinically infertile” particularly refers to the terms “infertile” or “sub-fertile” based on the standard medical classification: a woman is considered sub-fertile (or clinically infertile) if the woman does not or is likely to not conceive within a year of unprotected intercourse. In turn a high fertility status particularly refers to a woman that is likely to conceive at least within one year of unprotected intercourse.

The inventors were the first to realize that despite the complex interplay and synchronization of hormones required for conception, the evaluation of at least one physiological signal is sufficient to determine the fertility status of a woman, while detecting and measuring specific hormone concentrations at specific times is not necessary to provide a good estimation on the fertility status.

This in turn allows the user-friendly system with a wearable device according to the invention to non-invasively estimate the fertility status of a woman during daily life, particularly without the need to interrupt daily life routines for (invasive) hormone level determination.

According to another embodiment of the invention, the system is configured and arranged to record and/or store the sensor data from the at least one sensor continuously or intermittently over a period of time, such as days or months, particularly over the period of one or more menstrual cycles of the woman wearing the wearable device, wherein the system is configured and arranged to associate the sensor data to the time at which the sensor data have been generated such that a set of time-associated sensor data is generated, wherein the system is configured and arranged to store the set of time-associated sensor data, wherein the evaluation system is configured and arranged to classify the set of time-associated sensor data, e.g. the sensor data associated to the recording time, into at least a first group and a second group, particularly wherein the first group is associated to time-associated sensor data indicative of a woman having a high fertility status and wherein the second group is associated to time-associated sensor data indicative of a woman having a low fertility status.

According to this embodiment the sensor data are for example recorded at a sensor specific frequency, typically between 1 Hz and 50 Hz and stored as aggregate sensor data in a lower storage frequency such as 0.1 Hz. Such aggregate sensor data for example comprise the average sensor data values that have been recorded during one period of the storing frequency.

Therefore, the sensor data while being generated particularly almost continuously at a sensor specific frequency, the aggregate sensor data is stored at lower rates such that memory is conserved. The aggregate sensor data is then particularly used for classification.

According to another embodiment of the invention, the wearable device is configured and arranged to store the sensor data, particularly the aggregate sensor data of one night and to send the sensor data stored during this night to the evaluation system or to an external data storage.

The system particularly comprises a data storage configured and arranged to store the sensor data, particularly the time-associated sensor data.

The data storage is for example physically integrated in the wearable device and/or comprised by the evaluation system. Moreover, the data storage can be located on a yet different computer, or server, that is mechanically disconnect to the wearable device and the evaluation system. The data storage can be a cloud storage.

The set of time-associated sensor data can be displayed for example as a graph depicting the particularly aggregated sensor data in dependency of the time it was recorded.

Classification into the first and or the second group is particularly done once, particularly only when time-associated sensor data for at least 28 days, particularly a whole menstrual cycle, has been collected by the system.

Thus, according to this embodiment classification particularly is based on the evaluation of a temporal evolution of the sensor data during at least a portion of the menstrual cycle, at least one menstrual cycle or a plurality of menstrual cycles.

According to another embodiment of the invention, the evaluation system is configured and arranged to determine a set of normalized time-associated sensor data from the set of time-associated sensor data and to classify the set of normalized time-associated sensor data into at least the first or the second group, particularly wherein the set of normalized time-associated sensor data is a zero-mean sensor data set, particularly wherein the first group is associated to normalized time-associated sensor data indicative of a woman having a high fertility status and wherein the second group is associated to normalized time-associated sensor data indicative of a woman having a low fertility status.

Normalized time-associated sensor data exhibit particularly strong features that can be used for classification.

A zero-mean normalization of sensor data yields normalized sensor data that have an average value of zero. Zero-mean sensor data can be generated by subtracting the average value of all sensor data values from each sensor data value, generating sensor data values fluctuating around 0.

Zero-mean normalized sensor data are robust for classification.

According to another embodiment of the invention, the physiological signal is at least one of:
A temperature, particularly a skin temperature of the woman wearing the wearable device, particularly wherein the at least one sensor comprises a temperature sensor;
A conductance of the skin of the woman wearing the wearable device, particularly wherein the at least one sensor comprises a conductance or an impedance sensor;
A perfusion, particularly wherein the at least one sensor is an optical sensor configured and arranged to record a photoplethysmogram, particularly wherein the at least one sensor is a pulse oximeter
A heart rate, particularly wherein the at least one sensor is an optical sensor configured and arranged to record the heart rate,
A breathing rate, particularly wherein the at least one sensor is an optical sensor configured and arranged to record a breathing rate
A vascular activity.

Any one or a plurality of the physiological signals according to this embodiment can be used to estimate the fertility status of the woman wearing the wearable device.

Recording a plurality of physiological signals particularly yields sensor data that give rise to a more robust estimation of the fertility status, as statistical or erroneous variations and deviations of one physiological signal are compensated by the other physiological signals.

The terms “pulse” and “heart rate” are, in the context of this specification, used interchangeably.

The term “vascular activity” particularly refers to processes of the vascular system in response to or in connection with heart beating, such as pulse, blood flow, blood pressure, perfusion etc.

According to another embodiment of the invention, the at least one sensor is or comprises
A temperature sensor, such as a thermometer,
An optical sensor, particularly wherein the optical sensor comprises an infrared emitting light source configured to emit light in the wavelength region between 700 nm and 1500 nm, and/or wherein the light source is a green light emitting light source configured to emit light in the wavelength region between 500 nm to 560 nm,
A conductance sensor configured to record a skin conductance,
An impedance sensor configured to record a skin impedance.

According to another embodiment of the invention, the wearable device comprises or consists of a wrist-wearable sensor device, such as a watch or a smart watch, particularly wherein the at least one sensor is in contact with the skin of the woman wearing the wearable device.

According to another embodiment of the invention, the system, particularly the wearable device, comprises a motion detection sensor generating motion sensor data indicative of movement of the woman, wherein the system, particularly the evaluation system is configured to detect resting phases, particularly sleeping phases of the woman wearing the wearable device from the motion sensor data.

The motion sensor data are particularly provided to the evaluation system. A motion detection sensor is for example an inertial measurement unit (IMU), an accelerometer, or a GPS system configured to detect motion.

For detecting resting phases the evaluation system can comprise or have access to a timer module or a clock in order to identify resting times such as sleeping times. In addition, a breathing rate and/or a heart rate, for example recorded in the sensor data, can be used by the evaluation system to detect such resting phases, particularly in combination with the motion detection sensor.

According to another embodiment of the invention, the system is configured and arranged to detect resting phases, particularly sleeping phases of the woman wearing the device, and wherein the evaluation system is configured to use sensor data from the at least one sensor acquired during detected resting phases for classification, particularly wherein the evaluation system is configured to use exclusively sensor data acquired during detected resting phases, particularly wherein the system is configured to acquire sensor data solely during resting phases of the woman wearing the wearable device.

The detection of resting phases can for example be facilitated by means of or in combination with the motion detection sensor and its motion sensor data.

This embodiment allows a more robust classification as sensor data are acquired during the same physical state of the woman, particularly over the course of several days or months, and thus are not corrupted by a varying influence of the physical state on the sensor data.

According to another embodiment of the invention, the wearable device comprises a plurality of sensors for detecting a plurality of physiological signals, such as temperature, a skin conductance, a perfusion, a heart rate and/or a breathing rate, particularly wherein the evaluation system is configured to process the plurality of sensor data generated and provided from the plurality of sensors, and to classify the recorded sensors data to the first group or the second group.

The combination of a plurality of sensor signals from different physiological signals provides a more robust classification into the at least first or the second group.

Classification can be done for example by performing classification for sensor data from each sensor separately, or by processing the plurality of sensor data of all sensors in totality. Both approaches yield valid results.

Additionally, a fail-safe mechanism is provided in case one sensor malfunctions and provides corrupted sensor data.

According to another embodiment of the invention, the system, particularly the wearable device comprises a display configured to indicate whether the recorded sensors data are classified in the first or the second group.

The display can be on the wearable device or on a separate device such as a mobile device that is mechanically disconnect from the wearable sensor device.

According to another embodiment of the invention, the evaluation system comprises a trained classifier, such as a support vector machine that is used for classifying the recorded sensor data at least into the first group or the second group, or wherein the evaluation system comprises a machine learning module, such as an artificial neural network or a random forest classifier, trained to classify the recorded sensor data at least into the first group or the second group.

The trained classifier can be an electronic circuit, a processor or a computerized device configured to execute the classification of the sensor data, or any kind of processed, filtered or aggregated sensor data. The electronic circuit, the processor or the computerized device can be configured by means of a computer program that is executed on said devices.

The computer program is therefore configured to cause these devices to execute the computer program code facilitating the classification.

According to another embodiment of the invention, the evaluation system comprises, e.g. has stored or has access to a first model set of time-associated, particularly normalized sensor data associated to the first group and a second model set of time-associated, particularly normalized sensor data associated to the second group, wherein the system is configured to compare the sensor data, particularly the set of time-associated, particularly normalized sensor data to the first model set and the second model set and to classify the recorded sensor data into the first group or the second group, particularly based on a score value determined from a score function, wherein the score function is configured to determine a similarity between the recorded sensor data and the first and second model set of sensor data, particularly wherein the score function is a chi-square function or a mean square error between the recorded sensor data and the first or the second model set.

The first and or the second model sets are for example generated or acquired from sensor data from a population of fertile women (first group) and a population of clinically infertile women (second group).

The problem according to the invention is furthermore solved by a computer-implemented method for estimating a fertility status of a woman, particularly with a system according to any of the preceding claims, wherein the method comprises the steps of:
Recording at least one physiological signal from a woman with a sensor for recording a physiological signal,
Generating sensor data from the recorded physiological signal;
Classifying the sensor data into at least a first group or a second group, wherein the first group is associated to sensor data indicative of a woman having a high fertility status and wherein the second group is associated to sensor data indicative of a woman having a low fertility status.

It is noted that terms and definitions relating to the system according to the invention also apply to the method according to the invention and vice versa.

According to another embodiment of the invention, the method is executed on the system according to the invention.

According to another embodiment of the invention, the sensor data are recorded continuously or intermittently over a period of time, such as days or months, particularly over the period of one or more menstrual cycles of the woman wearing the wearable device, wherein the sensor data are associated to the time at which the sensor data have been generated such that a set of time-associated sensor data is generated, wherein the set of time-associated sensor data is stored, wherein the set of time-associated sensor data is classified into at least a first group or a second group, particularly wherein the first group is associated to time-associated sensor data indicative of a woman having a high fertility status and wherein the second group is associated to time-associated sensor indicative data of a woman having a low fertility status.

Storage of the sensor data can be facilitated on a data storage of the system.

According to another embodiment of the invention, a set of normalized time-associated sensor data is determined from the set of time-associated sensor data and classified into at least the first or the second group, particularly wherein the set of normalized time-associated sensor data is a zero-mean sensor data set, particularly wherein the first group is associated to normalized time-associated sensor data indicative of a woman having a high fertility status and wherein the second group is associated to normalized time-associated sensor data indicative of a woman having a low fertility status.

According to another embodiment of the invention, the at least one physiological signal is one of:
A temperature, particularly a skin temperature of the woman wearing the wearable device, particularly wherein the at least one sensor comprises a temperature sensor;
A conductance of the skin of the woman wearing the wearable device, particularly wherein the at least one sensor comprises a conductance or an impedance sensor;
A perfusion, particularly wherein the at least one sensor is an optical sensor configured and arranged to record a photoplethysmogram, particularly wherein the at least one sensor is a pulse oximeter
A heart rate, particularly wherein the at least one sensor is an optical sensor configured and arranged to record the heart rate,
A breathing rate, particularly wherein the at least one sensor is an optical sensor configured and arranged to record a breathing rate
A vascular activity.

According to another embodiment of the invention, resting phases, particularly sleep phases are detected particularly with the motion detection sensor and sensor data are evaluated for resting phases, particularly only for resting phases of the woman, particularly wherein the sensor data acquired during detected resting phases are used for classification, particularly wherein only sensor data acquired during detected resting phases are used for classification, particularly wherein sensor data are acquired solely during resting phases of the woman wearing the wearable device.

According to another embodiment of the invention, a trained classifier is employed to classify the sensor data into at least the first or into the second group, particularly wherein the classifier is a machine learning module, such as a support vector machine, a trained artificial neural network or another machine learning module.

According to another embodiment of the invention, a first model set of time-associated, particularly normalized sensor data associated to the first group and a second model set of time-associated, particularly normalized sensor data associated to the second group are provided, for example from a database, wherein the recorded sensor data, particularly the set of time-associated, particularly normalized sensor data are compared to the first model set and the second model set and classified into the first group or the second group, particularly based on a score value determined from a score function, wherein the score function is configured to determine a similarity between the recorded sensor data and the first and second model set of sensor data, particularly wherein the score function is a chi-square function or a mean square error between the recorded sensor data and the first or the second model set.

The problem according to the invention is furthermore solved by a computer program for executing the computer-implemented steps of the method according to the invention, wherein the computer program comprises computer program code configured to cause the system to execute the computer-implemented method according to the invention, when the computer program is executed on the system.

### Figure Description

Particularly, exemplary embodiments are described below in conjunction with the Figures. The Figures are appended to the claims and are accompanied by text explaining individual features of the shown embodiments and aspects of the present invention. Each individual feature shown in the Figures and/or mentioned in said text of the Figures may be incorporated (also in an isolated fashion) into a claim relating to the system or method according to the present invention.

FIG. 1 shows a schematic illustration of an electronic system according to the invention for detecting a plurality of physiological signals, the electronic system comprising a wearable device, in particular a wrist-worn bracelet, and an evaluation system comprising a processing system, an analysing system, a predicting system and a communication system in the wearable device and/or in an external system.

FIG. 2 shows a schematic illustration of an electronic system according to the invention for detecting a plurality of physiological signals, the electronic system comprising a wearable device, in particular a wrist-worn wearable sensor device associated with an electronic mobile device, and an evaluation system comprising a processing system, an analysing system, a predicting system and a communication system in the wearable device and/or in an external system;

FIG. 3 shows a schematic illustration of the wearable device;

FIG. 4 shows conductance of a group of fertile women and a group of sub-fertile women relative to the individual's cycle;

FIG. 5 shows perfusion measured with infrared of a group of fertile women and a group of sub-fertile women relative to the individual's cycle;

FIG. 6 shows perfusion measured with a green LED of a group of fertile women and a group of sub-fertile women relative to the individual's cycle;

FIG. 7 shows the skin temperature of a group of fertile women and a group of sub-fertile women;

FIG. 8 shows perfusion measured with infrared of a group of fertile women and a group of sub-fertile women;

FIG. 9 shows perfusion measured with a green LED of a group of fertile women and a group of sub-fertile women;

FIG. 10 shows conductance of a group of fertile women and a group of sub-fertile women;

FIG. 11 shows the breathing rate of a group of fertile women and a group of sub-fertile women; and

FIG. 12 shows the pulse rate of a group of fertile women and a group of sub-fertile women.

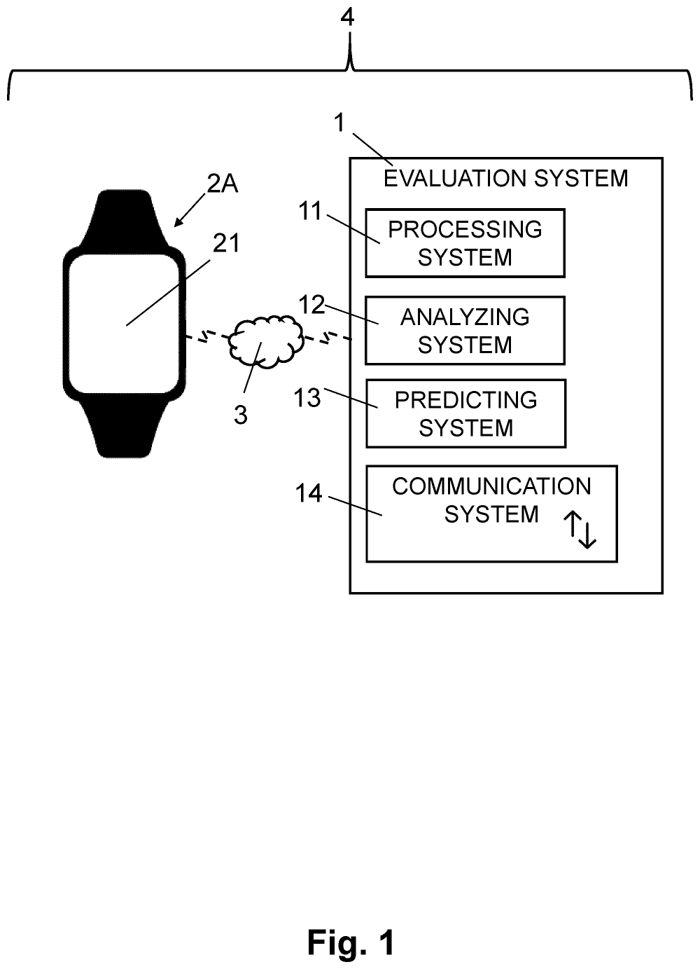

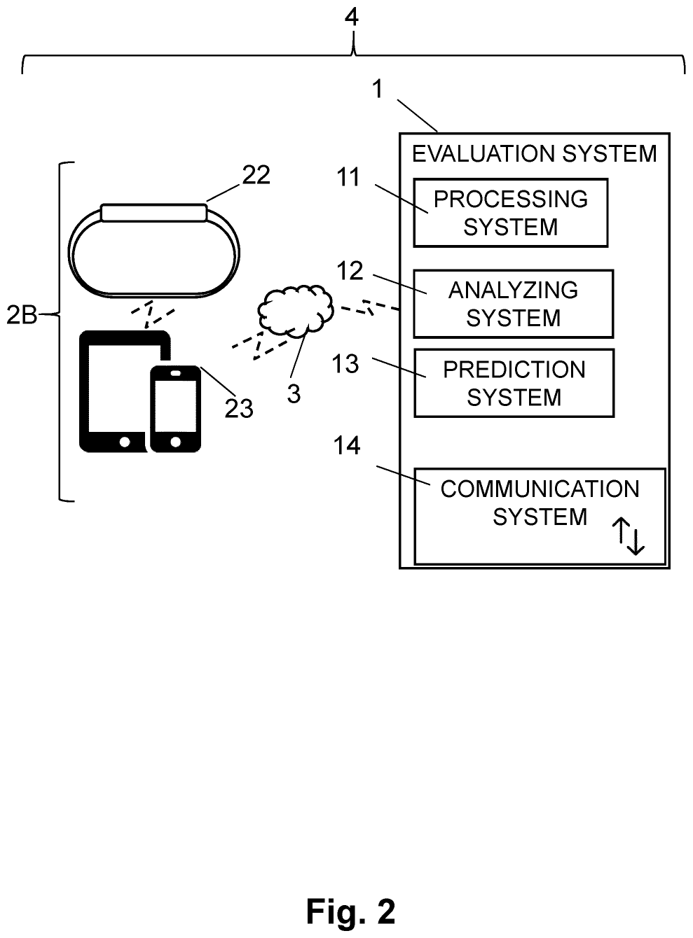

FIG. 1 schematically shows an embodiment of the system 4 according to the invention. The system 4 comprises a computerized evaluation system 1 comprising a processing system 11, an analysing system 12, a predicting or classification system 13, and a communication system 14 configured to receive sensor data from a wearable device 2A. The wearable device 2A can be a single device 2A or, as illustrated in FIG. 2, the device can comprise several particularly mobile devices 2B such as a smart watch 22, associated to an electronic mobile device 23, such as a tablet or mobile phone.

The wearable device 2A in FIG. 1 comprises a sensor device 21, here a smart watch, which has an integrated sensor system with at least one sensor (not shown) to record the physiological signal(s) of the woman wearing the smart watch. The wearable device 2A is configured for data communication with the evaluation system 1 that is mechanically disconnect from the wearable device 2A, i.e. the evaluation system 1 is arranged in a different housing and in a different location than the wearable device 2A. The evaluation system 1 can be hosted in a cloud, or on a cloud server, having access to databases or sensor data storage (not shown).

The evaluation system 1 is configured and arranged to perform the following steps: reception of sensor data from the wearable device 2A with the communication system 14, sensor data processing such as normalizing sensor data, receiving time-associated sensor data from a storage device, sensor data analysis, and estimation of the fertility status of the woman wearing the wearable device 2A. The evaluation system is further configured to allow communication with the wearable device 2A.

As illustrated in FIG. 2, in an alternative embodiment, the wearable device 2B comprises two devices 22, 23, one of which is a wearable sensor device 22 and wherein the other is a mobile electronic device 23 with a display. The wearable sensor device 22 is configured to be worn in close contact with the skin. It can be worn on the wrist, on the finger, on the arm, leg, foot, around the abdomen or the head. The wearable sensor device 22 communicates with the mobile electronic device 23, such as e.g. a mobile phone, a smart watch or tablet computer, for example via close range communication, WiFi or a mobile data network. For close-range communication, the wearable sensor device 22 and the mobile electronic device comprise a Bluetooth communication module (not shown), e.g. a Low Energy Bluetooth module, or another close-range communication module configured for direct data communication with the external mobile device 23.

As illustrated in FIG. 2, the mobile electronic device 23 is configured to facilitate the data communication between the wearable sensor device 22 and the evaluation system 1, e.g. by relaying the sensor data from the wearable sensor device 22 via a data network 3 to the remote evaluation system 1, for further processing.

Although not illustrated in FIGS. 1 and 2, the wearable sensor devices 21, 22 further comprise a timer module configured to generate current time and date information, e.g. a clock circuit or a programmed timer module. The timer module is further configured to generate time stamps including the current time and date, such that time-associated sensor data can be generated.

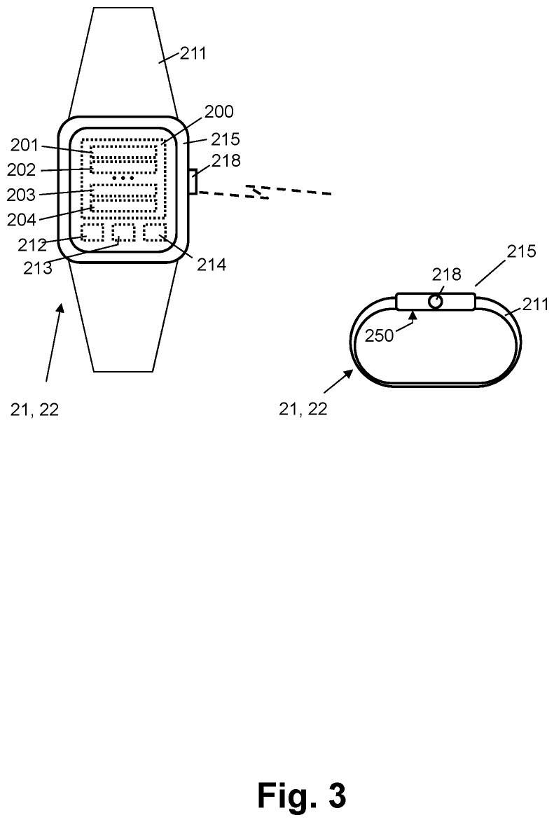

As illustrated schematically in FIG. 3, the wearable sensor device 21, 22 comprises several sensor systems 200 in a housing 215. Such sensor systems 200 comprises a first sensor system 201 with at least one optical sensor configured to generate photoplethysmography (PPG) signals for measuring heart signals, heart rate, heart rate variability, perfusion, and/or a breathing rate. For example, the first sensor system 201 comprises a PPG-based sensor system for measuring heart signals, heart rate and heart rate variability.

In yet another embodiment, the PPG signal is used to perform a pulse wave analysis.

According to the embodiment shown in FIG. 3, the sensor system 200 further comprises a second sensor system 202 with one or more motion detecting sensors such as accelerometers, for measuring body movements (acceleration). For the purpose of sleep phase detection, the motion sensor data from the accelerometers are processed in combination with the sensor data generated from the PPG-based sensor system.

The sensor system 200 in FIG. 3 further comprises a temperature sensor system 204 for measuring the temperature of the woman wearing the wearable sensor device 21, 22; specifically, the user's skin temperature; more specifically, the skin temperature of the wrist. The temperature sensor system 204 comprises one or more sensors, including at least one temperature sensor, and in an embodiment one or more additional sensor(s) for measuring further parameters like perfusion, bioimpedance and/or heat loss for determining the user's temperature.

Depending on the embodiment, the sensor systems 200 can further comprise a bioimpedance sensor system 203 with an electric impedance or conductance measuring system. The optical sensors of the first sensor system 201, the bioimpedance sensor system 203, and the temperature sensor system 204 are integrated in a housing 215 of the wearable sensor device 21, 22 and are arranged on a rear side 250 of the wearable sensor device 21, 22, facing the user's skin in a mounted state of the wearable sensor device 21, 22.

In the mounted state, when the wearable sensor device 21, 22 is actually worn, e.g. on the wrist, just as one would wear a watch, the rear side 250 of the wearable sensor device 21, 22 or the rear side 250 of its housing 215, respectively, is in contact with the skin, e.g. the skin of the wrist. The optical sensors of the first sensor system 201, the bioimpedance system 203, and the temperature sensor system 204 touch the skin or at least face the skin, e.g. the skin of the wrist.

The wearable sensor device 21, 22 further comprises a data storage 212, e.g. a data memory such as RAM or flush memory, and an operational processor 213 connected to the data storage 212 and the sensor systems 200.

The wearable sensor device 21, 22 further comprises a communication system 214 connected to the processor 213. Depending on the embodiment, the communication system 214 is configured for data communication with a separate external system 1, as illustrated in FIG. 1, or with a mobile electronic device 23, as illustrated in FIG. 2. Accordingly, the communication system 214 is configured for data communication via a close-range communication interface or other data communication networks. For example, for close range communication, the communication system 214 comprises a Bluetooth communication module, e.g. a Low Energy Bluetooth module, or another close-range communication system configured for direct data communication with the external mobile communication device 23.

The data network 3 comprises a mobile radio network such as a GSM-network (Global System for Mobile communication), a UMTS-network (Universal Mobile Telephone System), or another mobile radio telephone system, a wireless local area network (WLAN), and/or the Internet.

As further illustrated in FIG. 3, the wearable sensor device 21, 22 further comprises one or more data entry elements 218 enabling the user to enter data and/or event indications. Depending on the embodiments, data entry elements 218 comprise data entry buttons, keys and/or rotary selection switches. The wearable sensor device 21, 22 is worn in close contact to the skin. The strap 211 can for example be a band, a cuff, a bracelet, an elastic rubber band, a head band, a ring, or a belt.

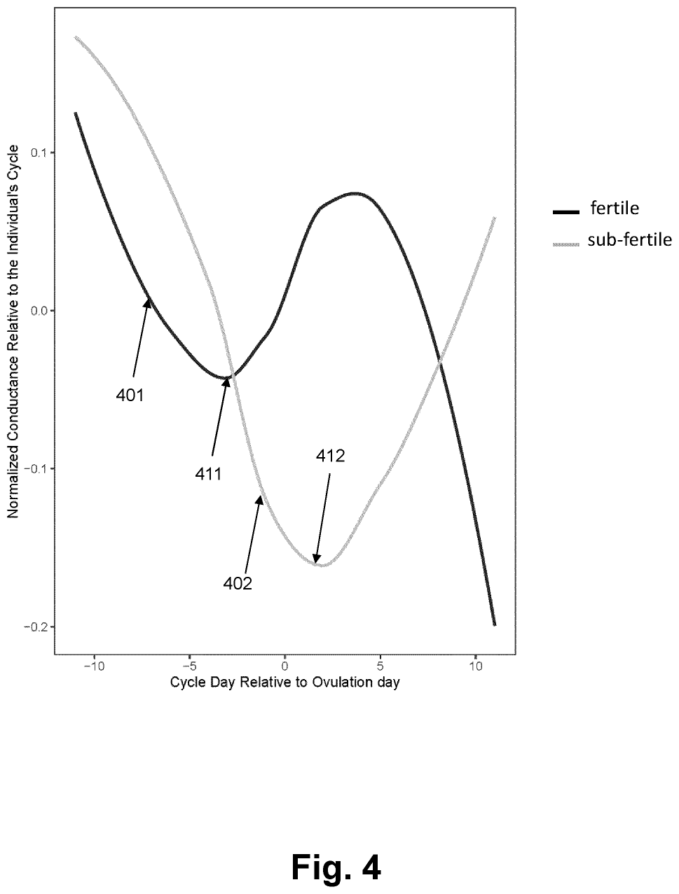

FIG. 4 shows a first model set of time-associated normalized sensor data 401 for conductance for a first group comprising women having a high fertility status and a second model set of time-associated normalized sensor data 402 for conductance for a second group comprising sub-fertile women, i.e. having a low fertility status. The sensor data of the women from the first group have been averaged as well as the sensor data of the women from the second group in order to generate the model data sets 401, 402. The first and the second model data set are normalized to the average value for the cycle, such that the sensor data values are transformed to zero-mean values data sets. The model sets of normalized sensor data for conductance significantly differ between the first group of high fertility status and the second group of sub-fertile women. The temporal evolution for the first group shows a local minimum 411 during late luteal phase of the menstrual cycle, while for the second group a local minimum 412 was observed around ovulation during the menstrual cycle.

Data were acquired during an observational study that analysed changes in physiological signals, such as resting pulse rate, heart rate variability features, breathing rate, temperature, perfusion, and conductance for a total of 268 menstrual cycles of 77 women. The menstrual cycles and the measured physiological signals as well as sensor data were classified as fertile (first group) or sub-fertile (second group) based on the standard medical classification: a couple is considered sub-fertile (or clinically infertile) if it does not conceive within a year of active unprotected intercourse.

To that end, 34 women did not conceive during the observation (second group, sub-fertile), and 33 women got pregnant during the study (first group, fertile). For the second group, all menstrual cycles were included in the analysis, for the first group, the menstrual cycles when conception took place were excluded, as the behaviour of the physiological signals changes significantly after conception.

In order to estimate the fertility status of an individual woman, acquired time-associated sensor data for the conductance during the menstrual cycle of this woman are provided to the evaluation system, where the time-associated sensor data might be processed, e.g. normalized by the processing system 11, the analysing system 12 performs an analysis comparing the set of acquired time-associated sensor data generated from the physiological signals of the woman and the first and second model sets of time-associated sensor data for conductance of the first and the second group followed by a classification to the first group or the second group depending to which model set a higher degree of similarity can be established.

The communication system 14 communicates to the wearable device 2A, 2B e.g. a mobile electronic device 23, smart phone, or tablet computer, that the woman's chances of getting pregnant are high, i.e. that the woman has a high fertility status, if the classification found a higher degree of similarity to the first model set or that the woman's chances of getting pregnant are low, i.e. that the woman has a low fertility status, if the classification found a higher degree of similarity to the second model set.

If classification is associated with a degree of uncertainty, information about the degree of uncertainty can be transmitted to the wearable device as well.

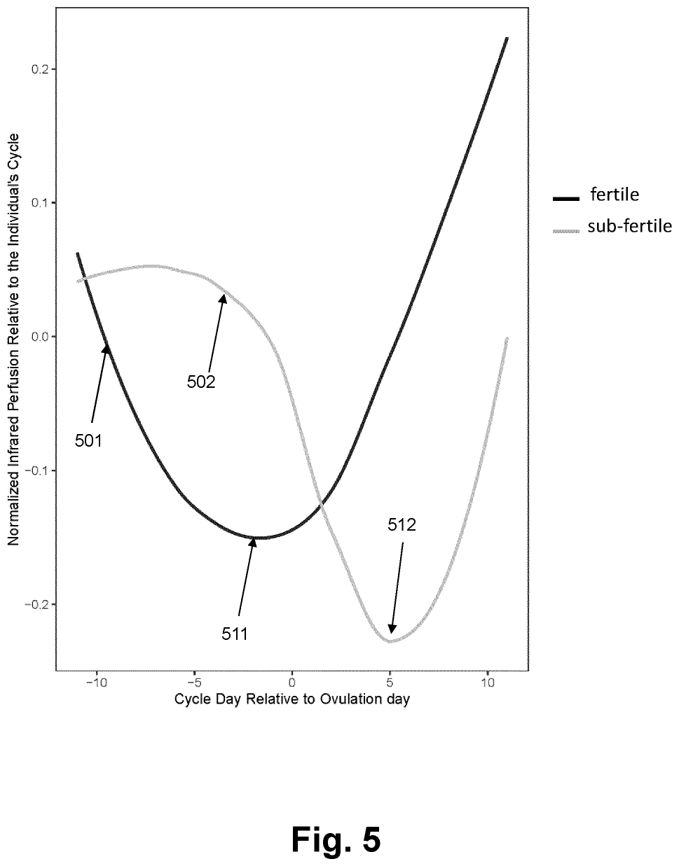

FIG. 5 shows the first and the second normalized model sets 501, 502 for perfusion measured with a PPG sensor utilizing an infrared LED. The values were normalized to the individual average value for the menstrual cycle. The local minimum 511 of the graph for the first model set 501 associated to the first group was around the time of ovulation, while the local minimum 512 of the function of graph for the second model set 502 associated to the second group can be observed 5 days later, i.e. in early luteal phase.

Therefore, estimation of the fertility status can be performed also based on PPG sensor data acquired with an infrared emitting LED for example by identifying the time of occurrence of the local minimum 511, 512.

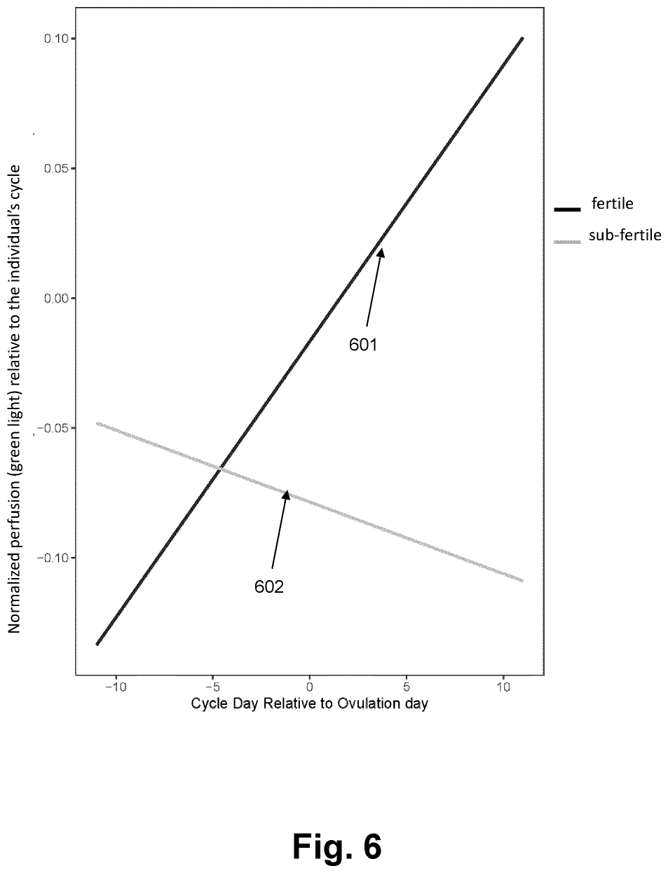

FIG. 6 shows the normalized values 601, 602 for perfusion measured with a PPG sensor utilizing a green LED. The gradient of the function of the sub-fertile group was significantly smaller than the gradient of the function of the fertile group.

In another embodiment of the invention, the system measures perfusion with an infrared LED (perfusion IR) and/or a green LED (perfusion green) during the menstrual cycle of a woman, the physiological signals are processed in the evaluation system 1, the analysing system 12 performs an analysis comparing the set of acquired time-associated sensor data generated from the perfusion signal of the woman and the first and second model sets of time-associated sensor data for perfusion of the first and the second group followed by a classification to the first group or the second group depending to which model set a higher degree of similarity can be established.

The communication system 14 communicates to the wearable device 2A, 2B e.g. a mobile electronic device 23, smart phone, or tablet computer, that the woman's chances of getting pregnant are high, i.e. that the woman has a high fertility status, if the classification found a higher degree of similarity to the first model set or that the woman's chances of getting pregnant are low, i.e. that the woman has a low fertility status, if the classification found a higher degree of similarity to the second model set.

If classification is associated with a degree of uncertainty, information about the degree of uncertainty can be transmitted to the wearable device as well.

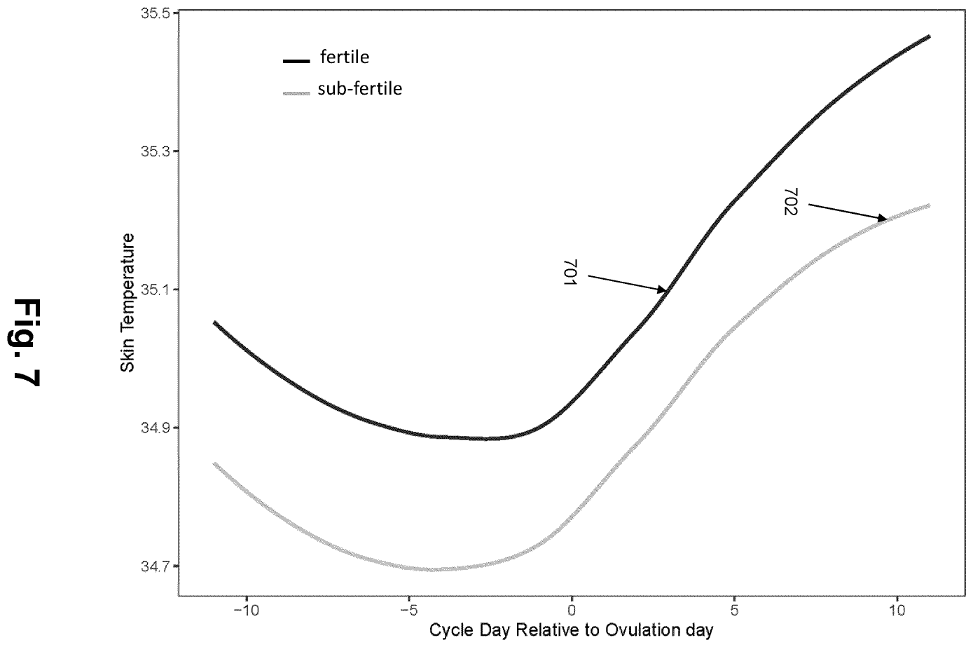

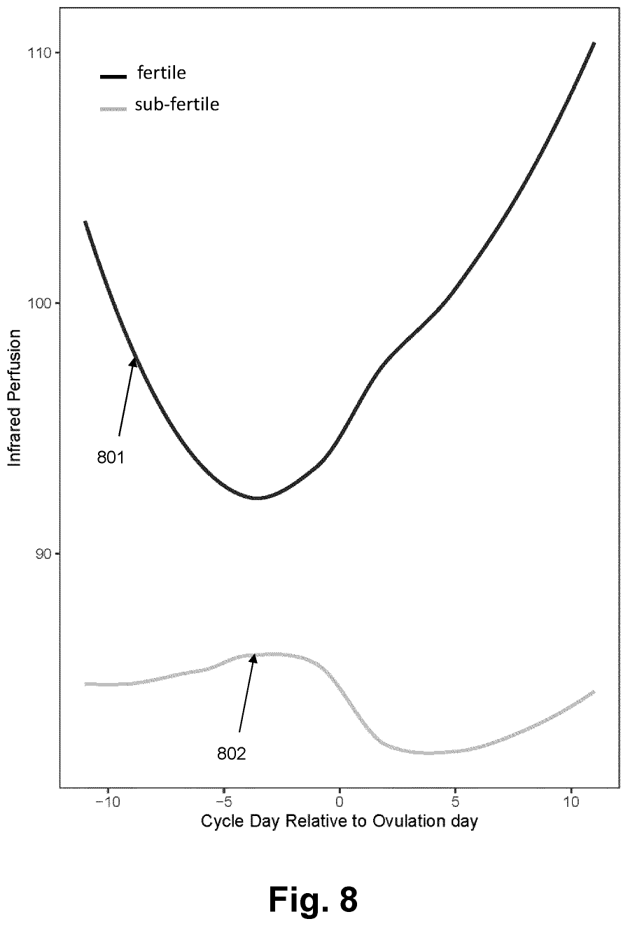

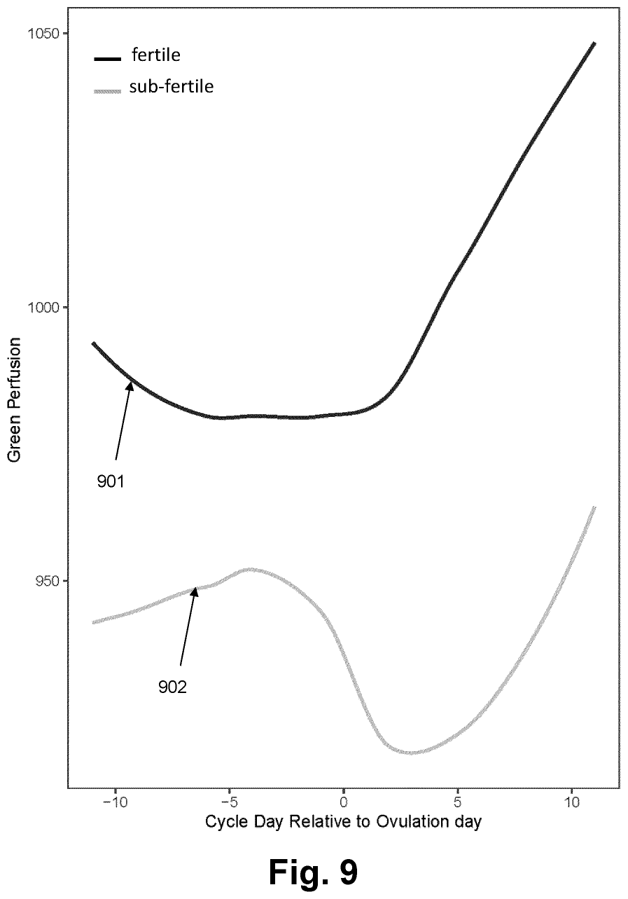

In FIGS. 7, 8 and 9, the comparison of the absolute values for temperature 701 (data of first group), 702 (data of second group), perfusion 801 (data of first group), 802 (data of second group) measured with infrared and perfusion 901 (data of first group), 902 (data of second group) measured with green light show that the sensor data values of the second group are lower throughout the menstrual cycle compared to the sensor data from the first group for all physiological signal acquired.

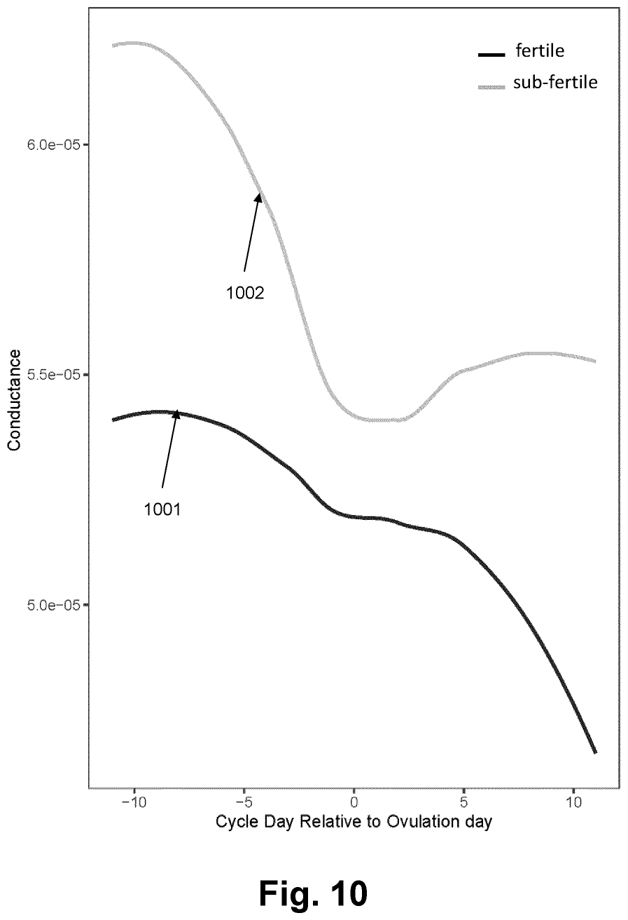

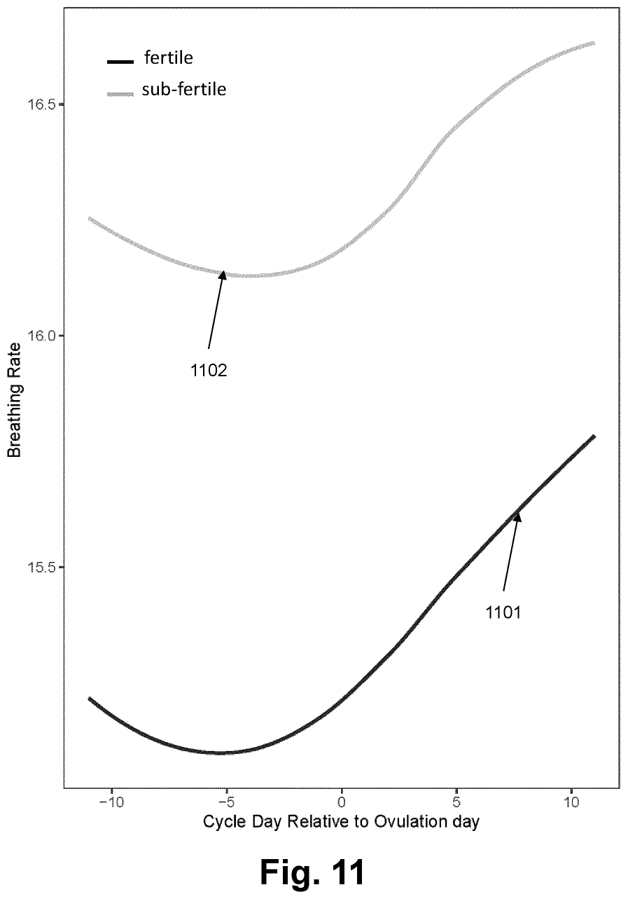

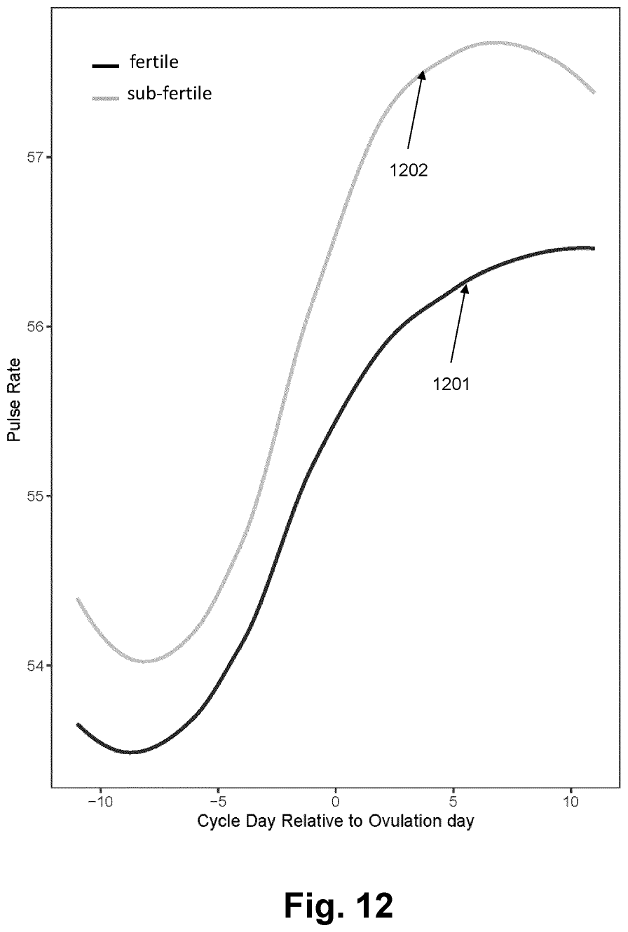

FIGS. 10, 11 and 12 show that the absolute values for breathing rate 1101 (data of first group), 1102 (data of second group), heart rate 1201 (data of first group), 1202 (data of second group), and conductance 1001 (data of first group), 1002 (data of second group), in the second group are higher throughout the menstrual cycle compared to the sensor data from the first group for these physiological signals.

Therefore, the invention allows an estimation of the fertility status by evaluating some, all or just one of the physiological signals as described.

1. A system for estimating a fertility status of a woman, particularly for determining a conception probability of a woman, the system comprising:
a wearable device, comprising at least one sensor configured to record at least one physiological signal from a woman wearing the wearable device and to generate sensor data from the at least one physiological signal, wherein the wearable device is configured and arranged to provide the sensor data to
an evaluation system configured and arranged to receive and process the sensor data from the wearable device, wherein the evaluation system is further configured and arranged to classify the sensor data into at least a first group and a second group, wherein the first group is associated to sensor data indicative of a woman having a high fertility status and wherein the second group is associated to sensor data indicative of a woman having a low fertility status.

2. The system according to claim 1, wherein the system is configured and arranged to record the sensor data from the at least one sensor continuously or intermittently over a period of time, such as days or months, particularly over the period of one or more menstrual cycles of the woman wearing the wearable device, wherein the system is configured and arranged to associate the sensor data to the time at which the sensor data have been generated such that a set of time-associated sensor data is generated, wherein the system is configured and arranged to store the set of time-associated sensor data, wherein the evaluation system is configured and arranged to classify the set of time-associated sensor data into at least a first group or a second group, particularly wherein the first group is associated to time-associated sensor data indicative of a woman having a high fertility status and wherein the second group is associated to time-associated sensor data indicative of a woman having a low fertility status.

3. The system according to claim 1 or 2, wherein the evaluation system is configured and arranged to determine a set of normalized time-associated sensor data from the set of time-associated sensor data and to classify the set of normalized time-associated sensor data into at least the first or the second group, particularly wherein the set of normalized time-associated sensor data is a zero-mean sensor data set, particularly wherein the first group is associated to normalized time-associated sensor data indicative of a woman having a high fertility status and wherein the second group is associated to normalized time-associated sensor data indicative of a woman having a low fertility status.

4. The system according to claim 1 or 2, wherein the physiological signal is at least one of:
a temperature, particularly a skin temperature of the woman wearing the wearable device, particularly wherein the at least one sensor comprises a temperature sensor;
a conductance of the skin of the woman wearing the wearable device, particularly wherein the at least one sensor comprises a conductance or an impedance sensor;
a perfusion, particularly wherein the at least one sensor is an optical sensor configured and arranged to record a photoplethysmogram, particularly wherein the at least one sensor is a pulse oximeter
a heart rate, particularly wherein the at least one sensor is an optical sensor configured and arranged to record the heart rate,
a breathing rate, particularly wherein the at least one sensor is an optical sensor configured and arranged to record a breathing rate
a vascular activity.

5. The system according to claim 1 or 2, wherein the at least one sensor is or comprises
a temperature sensor such as a thermometer,
an optical sensor, particularly wherein the optical sensor comprises an infrared emitting light source configured to emit light in the wavelength region between 700 nm and 1500 nm, and/or wherein the light source is a green light emitting light source configured to emit light in the wavelength region between 500 nm to 560 nm,
a conductance sensor configured to record a skin conductance,
an impedance sensor configured to record a skin impedance.

6. The system according to claim 1 or 2, wherein the wearable device is a wrist-wearable sensor device, such as a watch or a smart watch, particularly wherein the at least one sensor is in contact with the skin of the woman wearing the wearable device.

7. The system according to claim 1 or 2, wherein the system, particularly the wearable device, comprises a motion detection sensor generating motion sensor data indicative of movement of the woman, wherein the system, particularly the evaluation system is configured to detect resting phases, particularly sleeping phases of the woman wearing the wearable device from the motion sensor data.

8. The system according to claim 1 or 2, wherein the system is configured and arranged to detect resting phases, particularly sleeping phases of the woman wearing the device, and wherein the evaluation system is configured to use sensor data from the at least one sensor acquired during detected resting phases for classification, particularly wherein the evaluation system is configured to use exclusively sensor data acquired during detected resting phases, particularly wherein the system is configured to acquire sensor data solely during resting phases of the woman wearing the wearable device.

9. The system according to claim 1 or 2, wherein the evaluation system comprises a trained classifier trained to classify the recorded sensor data at least into the first group or the second group, particularly wherein the classifier is a machine learning module, such as a support vector machine, a trained artificial neural network or a random forest classifier

10. The system according to claim 1 or 2, wherein the evaluation system comprises a first model set of time-associated, particularly normalized sensor data associated to the first group and a second model set of time-associated, particularly normalized sensor data associated to the second group, wherein the evaluation system is configured to compare the sensor data, particularly the set of time—associated, particularly normalized sensor data to the first model set and the second model set and to classify the recorded sensor data into the first group or the second group, particularly based on a score value determined from a score function, wherein the score function is configured to determine a similarity between the recorded sensor data and the first and second model set of sensor data, particularly wherein the score function is a chi-square function or a mean square error between the recorded sensor data and the first or the second model set.

11. A computer-implemented method for estimating a fertility status of a woman, particularly with a system according to any of the preceding claims, wherein the method comprises the steps of:
recording at least one physiological signal with at least one sensor from a woman,
generating sensor data from the recorded physiological signal;
classifying the sensor data into at least a first group or a second group, wherein the first group is associated to sensor data indicative of a woman having a high fertility status and wherein the second group is associated to sensor data indicative of a woman having a low fertility status.

12. The method according to claim 11, wherein the sensor data are recorded continuously or intermittently over a period of time, such as days or months, particularly over the period of one or more menstrual cycles of the woman wearing the wearable device, wherein the sensor data are associated to the time at which the sensor data have been generated such that a set of time—associated sensor data is generated, wherein the set of time-associated sensor data is stored, wherein the set of time-associated sensor data is classified into at least a first group and a second group, particularly wherein the first group is associated to time-associated sensor data indicative of a woman having a high fertility status and wherein the second group is associated to time-associated sensor indicative data of a woman having a low fertility status.

13. The method according to claim 11 or 12, wherein resting phases, particularly sleep phases are detected and sensor data are evaluated for resting phases, particularly only for resting phases of the woman, particularly wherein the sensor data acquired during detected resting phases are used for classification, particularly wherein only sensor data acquired during detected resting phases are used for classification, particularly wherein sensor data are acquired solely during resting phases of the woman wearing the wearable device.

14. The method according to claim 11 or 12, wherein a trained classifier is employed to classify the sensor data into at least the first or into the second group, particularly wherein the classifier is machine learning module, such as a support vector machine, a trained artificial neural network or a random forest classifier.

15. The method according to claim 11 or 12, wherein a first model set of time-associated, particularly normalized sensor data associated to the first group and a second model set of time-associated, particularly normalized sensor data associated to the second group are provided, wherein the recorded sensor data, particularly the set of time-associated, particularly normalized sensor data are compared to the first model set and the second model set and classified into the first group or the second group, particularly based on a score value determined from a score function, wherein the score function is configured to determine a similarity between the recorded sensor data and the first and second model set of sensor data, particularly wherein the score function is a chi-square function or a mean square error between the recorded sensor data and the first or the second model set.
